(19) United States
(12) Patent Application Publication
(10) Pub. No.: US 2016/0313317 A1
(43) Pub. Date: Oct. 27, 2016

(54) BIOMOLECULAR DETECTION USING MULTIPHOTON PLASMONIC COOPERATIVE COUPLING

(71) Applicant: EMPIRE TECHNOLOGY DEVELOPMENT LLC, Wilmington, DE (US)

(72) Inventor: Mattias Lennart ASLUND, Seattle, WA (US)

(73) Assignee: EMPIRE TECHNOLOGY DEVELOPMENT LLC, Wilmington, DE (US)

(21) Appl. No.: 15/026,070

(22) PCT Filed: Sep. 30, 2013
(86) PCT No.: PCT/US13/62633

§ 371 Date: Jul. 15, 2016

(51) Int. Cl. G01N 33/543; G01N 21/64

(52) U.S. Cl. CPC G01N 33/54373; G01N 33/54393; G01N 33/54346; G01N 21/6428; G01N 21/648

(57) ABSTRACT

Methods, systems, and kits are disclosed for determining the presence of a biomolecule. An assay plate may be composed of nanostructures capable of binding the biomolecule. Reporter species may be associated with the nanostructure-bound biomolecules. The assay plate may be illuminated with radiation capable of inducing plasmonic fields near the nanostructures. The fields may cause nearby reporter species to fluoresce. Nanostructures may be fabricated by illuminating nanoparticles in the presence of linker species. The induced plasmonic fields may cause the linker species to bind to the nanoparticles at field hotspots. Binder species conjugated with the linker species may form capture species capable of binding the biomolecule. A system for measuring the presence of a biomolecules may include a chamber that may be light tight, a source of illumination, and a photodetector. A kit for such a system may include an assay plate and solutions for the assay.

BACKGROUND

Current biomedical assays of ultra-high detection sensitivity and specificity may depend on major capital equipment for in-vitro diagnostic (IVD) purposes. These devices may be highly complex, very sensitive to environmental perturbations, and require expert staff to both operate and analyze the resulting data. The development of novel diagnostic devices that are more sensitive and less costly than those presently available, along with specific assays with simpler procedures and less read-out ambiguity, make up a major research area in the industry.

It is envisioned that the diagnostic devices and test kits may be made inexpensively and easy to use so that they can be used by individual patients at their homes. At present, the home-market is essentially limited to simple pregnancy tests and glucose-sensors. In addition, desirable over-the-counter tests may include assays for the most common cancer markers, heritable genetic disorders, predispositions for vascular diseases, historical genetic heraldry, environmental allergens (for example, traces of peanuts in food), pathogens, toxins, auto immune disease markers, and tests for drug use by children. Highly sensitive, low-cost screening assays may also be of value in livestock screening for contagious diseases, monitoring of food preparation processes, environmental pollution monitoring, as well as security and defense applications.

A key factor for the development of these markets may include the development of a device that relies on an inexpensive, ultra-sensitive method that may exhibit minimal error sources as well as being suitable for automated assaying and workup procedures. It is therefore desirable to develop such devices and methods.

### SUMMARY

In an embodiment of a method for detecting one or more biomolecules, the method may include providing a chamber having at least one photodetector, providing, within the chamber, at least one assay plate having multiple nanostructures including a plurality of biomolecule capture species, contacting the assay plate in the chamber with at least one sample solution suspected of containing the one or more biomolecules, contacting the assay plate in the chamber with an assay solution having a plurality of biomolecule reporter species, in which the plurality of biomolecule reporter species, when bound to the one or more biomolecules associated with the plurality of biomolecule capture species, is configured to emit at least one emission wavelength of radiation, placing the chamber into a light-tight state, providing, within the chamber, at least one source of electromagnetic radiation designed to produce electromagnetic radiation having at least one excitation wavelength greater than the at least one emission wavelength, exposing at least a portion of the nanostructures on the assay plate to the electromagnetic radiation, thereby inducing at least one plasmon dipole electric field adjacent to the exposed nanostructures, and detecting, by the at least one photodetector, the presence of the at least one emission wavelength.

In an embodiment of a method for fabricating an assay plate for detecting a biomolecule, the method may include providing a plate structure having a plurality of nanoparticles contacting a substrate, placing the plate structure in a chamber, the chamber including at least one illumination source, contacting the plate structure within the chamber with a first solution including at least one species of a molecular linker, placing the chamber into a light-tight state, exposing the plate structure to electromagnetic radiation emitted by the at least one illumination source, thereby inducing at least one plasmon dipole electric field adjacent to one or more of the plurality of nanoparticles, forming a plurality of nanoparticle/molecular linker complexes adjacent to at least a maximum region in the plasmon dipole electric field, and contacting the plurality of nanoparticle/molecular linker complexes with a second solution having at least one species of biomolecule binder, thereby forming the assay plate having a plurality of nanoparticle-linked biomolecule capture species disposed on the one or more nanoparticles at one or more locations of the induced at least one plasmon dipole electric field.

In an embodiment of a system for detecting one or more biomolecules, the system may be composed of at least one assay plate including a plurality of nanostructures contacting a substrate, in which at least a portion of the plurality of nanostructures has a biomolecule capture species configured to bind to the biomolecule, and a chamber having at least one illumination source and at least one photodetector, in which the chamber is configurable to be placed in a light-tight state, and the at least one illumination source is configured to emit electromagnetic radiation having at least one excitation wavelength designed to induce a plasmon dipole electric field adjacent to one or more of the plurality of nanostructures.

In an embodiment of a kit for use in a system for detecting one or more biomolecules, the kit may be composed of at least one assay plate including a plurality of nanostructures contacting a substrate, in which the plurality of nanostructures includes a plurality of pairs of nanoparticles having a first nanoparticle with a shape including at least one vertex and a second nanoparticle having a shape including at least one vertex, a source of a first solution including at least one binder species configured to bind to the one or more biomolecules, a source of a second solution including at least one molecular linker species, in which the molecular linker species has a first end configured to contact at least one of the plurality of nanostructures and a second end configured to contact the at least one binder species, and in which the first end of the molecular linker species is configured to contact the at least one of the plurality of nanostructures when the at least one of the plurality of nanostructures is illuminated by electromagnetic radiation, and at least one source of a third solution including a reporter species configured to bind to the one or more biomolecules.

### BRIEF DESCRIPTION OF THE DRAWINGS

FIG. 1 illustrates an example of an assay plate in accordance with the present disclosure.

FIGS. 2A and 2B illustrate examples of plasmon dipole fields that may be induced in nanoparticles in accordance with the present disclosure.

FIG. 3A is a flow chart illustrating an example of a method for detecting the presence of a biomolecule associated with an assay plate in accordance with the present disclosure.

FIGS. 3B-F illustrate an example of the method disclosed in FIG. 3A in accordance with the present disclosure.

FIG. 4A is a flow chart illustrating an example of a method for fabricating an assay plate in accordance with the present disclosure.

FIGS. 4B-F illustrate an example of the method disclosed in FIG. 4A in accordance with the present disclosure.

FIG. 5 illustrates an example of a system for detecting the presence of a biomolecule associated with an assay plate in accordance with the present disclosure.

FIG. 6 illustrates an example of a plate enclosure for use in a system for detecting the presence of a biomolecule associated with an assay plate in accordance with the present disclosure.

### DETAILED DESCRIPTION

Among the variety of available assay methods, sorbent methods such as ELISA (enzyme-linked immunosorbent assay) are well regarded for their specificity as well as potential sensitivity. A typical ELISA includes a multi-well plate in which the wells contain immobilized capture species capable of binding to one or more specific biomolecule targets. The assay may be performed by exposing the plate to a sample solution that may contain some amount of the biomolecular target. Excess sample may be washed away, resulting in some amount of the target biomolecules being bound to the capture species. The assay may be developed by exposing the plate, including any bound biomolecules, to a solution containing one or more reporter species. These species may include a second type of binding material linked to a fluorescent reporter molecule or a dye producing enzyme capable of converting an inactive reporting species to a detectable form. After the wells are exposed to the reporter species, the wells may be washed again to remove excess reporter species. The remaining conversion enzymes are expected to be bound only to the target biomolecules. The plates may then be exposed to a solution containing the substrate for the reporter species, which may be converted into a detectable species. In some examples, the detectable species may be detected by the amount of radiation that is absorbed at a specific wavelength. In another example, the detectable species may be one that fluoresces at a specific wavelength of electromagnetic radiation after excitation at a different wavelength.

A variety of technical issues may reduce the effectiveness of fluorescence-based ELISA assays. Although the reporter species may be designed to preferentially bind to the captured target biomolecule, non-specific binding to the well surface may also occur. For enzyme based immunosorbent assays, the amount of detectable species may depend on the enzymatic action including incubation conditions. Control of these conditions may be difficult to maintain for a home-based test kit.

Alternatives to the enzyme-based ELISA assay may include plasmonic resonance enhanced fluorescent immuno-assays (PREFI assay). In a PREFI assay, capture species are associated with an assay plate composed of a number of small, conductive nanostructures. These combined biomolecule-nanostructure species, when illuminated by electromagnetic radiation of suitable wavelength, may absorb and subsequently emit radiation associated with a plasmonic dipole field near the nanostructures. Fluorophores exposed to excitation radiation may fluoresce greater in the presence of the plasmonic electric field of the nanostructures than without such a field. Components of the energy flux may flow from the plasmonic field to the fluorescent marker or vice versa, in both the absorption and the emission phases. A fluorophore conjugated to a reporter species and immobilized by a sample bound to a capture species near the nanostructures may fluoresce strongly when the nanostructures transfer energy from a dielectric plasmonic field. The PREFI assay may demonstrate great sensitivity since the fluorescence output of the fluorophores may be enhanced due to the plasmonic dipole field. As a result, a smaller number of sample biomolecules may be detectable. The PREFI assay, as disclosed above, may be hampered significantly by two key issues. First, the capture species may be bound anywhere on the assay plate and not necessarily within the plasmon dipole field. The plate-bound capture species may create significant background noise and may also isolate virtually all target biomolecules in positions far from the plasmonic fields. Isolation of the fluorophores away from the plasmonic dipole fields may defeat the purpose of having high detection capacity (including a possibility of single molecule detection) for low concentration analytes. A second issue may include unwanted light noise, particularly due to scattered excitation light and bulk auto-fluorescence. For many fluorophores, the excitation wavelength is close to the fluorescence wavelength band, and it may be difficult and expensive to filter the scattered excitation light from the fluorescence emission wavelengths.

These issues may be addressed by methods to fix the capture species on the nanostructures, such as at the regions of maximum plasmonic dipole field (one or more plasmonic “hotspots”). Additionally, because the plasmonic dipole field may concentrate such a large amount of energy in a confined special location, multi-photon excitation processes may be used to excite fluorophores within the dielectric field with wavelength energies well below the normal excitation energy for the fluorophore. As a result, the fluorescence excitation wavelength energy may be well separated from the emission wavelength energy, and thus easier to filter. The methods, devices, and kits disclosed below may include one or more of these features to improve the effectiveness and robustness of such PREFI assays.

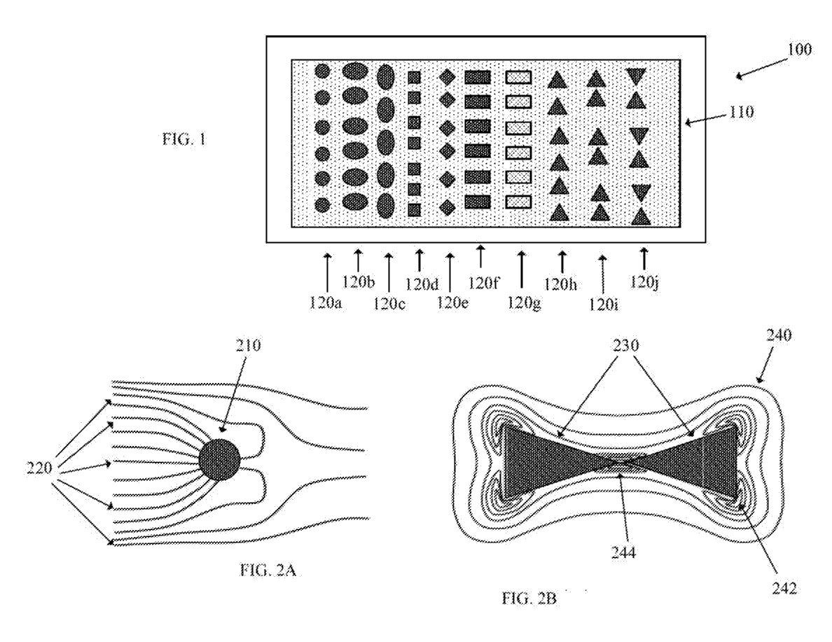

FIG. 1 illustrates an example of a PREFI assay plate 100. The assay plate 100 is composed of a number of nanostructures 120a-j associated with a substrate 110. The substrate 110 may include any dielectric material, including, but not limited to, one or more of a silicon, a glass, a ceramic, and a plastic. Non-limiting examples of a glass include silicon dioxide and soda lime glass. Additional non-limiting examples of a substrate 110 may include ceramics having a non-porous surface coating such as glass or a polymer. Non-limiting examples of a plastic may include Poly (methyl methacrylate) (PMMA), polycarbonate, and polystyrene.

The nanostructures 120a-j may be composed of one or more of a metal, a metal alloy, a semiconductor, and a carbon nanostructure. Non-limiting examples of metals may include silver, gold, copper, aluminum, platinum, palladium, and nickel. Non-limiting examples of metal alloys may include one or more of a gold-cadmium alloy, a silver-cadmium alloy, a gold-zinc alloy, a silver-zinc alloy, a gold/Group I alkali metal alloy, and a silver/Group I alkali metal alloy. Non-limiting examples of semiconductors, doped or undoped, may include one or more of silicon, gallium arsenide, gallium nitride, gallium phosphide, silicon carbide, indium-tin-oxide, aluminum-zinc-oxide, and gallium-zinc-oxide. A non-limiting example of a carbon nanostructure may include graphene.

The nanostructures 120a-j may have an average length of about 30 nanometers to about 1000 nanometers. Non-limiting examples of the average length of a nanostructure may include about 30 nanometers, about 50 nanometers, about 100 nanometers, about 200 nanometers, about 300 nanometers, about 400 nanometers, about 500 nanometers, about 600 nanometers, about 700 nanometers, about 800 nanometers, about 900 nanometers, about 1000 nanometers, and ranges between any two of these values including endpoints.

The nanostructures 120a-j may be composed of single nanoparticles. Non-limiting examples of such single nanoparticles are illustrated by nanostructures 120a-120h. Non-limiting examples of the shapes of such single nanoparticles may include a sphere, an ovoid, a section of a triangular prism 120h, a section of a rectangular prism 120d,e, a section of a cylindrical prism 120a, a section of a prism having a curved periphery 120b,c, a sheet 120f, a perforated sheet 120g in which the nanoparticles may be disposed in the vicinity of the perforations, and a section of a prism having an irregular periphery.

In an alternative embodiment, the nanostructures 120a-j may be composed of multiple nanoparticles forming clusters. In one non-limiting example of such a clusters, the nanostructures may be composed of pairs of nanoparticles 120i,j. It may be appreciated that each nanoparticle of the pairs of nanoparticles 120ij may have an independent shape, non-limiting examples including a sphere, an ovoid, a section of a triangular prism 120i,j, a section of a rectangular prism, a section of a cylindrical prism, a section of a prism having a curved periphery, a sheet, a perforated sheet, and a section of a prism having an irregular periphery. The shape of the first nanoparticle of the paired nanoparticles may be the same as that of the second nanoparticle. Alternatively, the shape of the first nanoparticle of the paired nanoparticles may differ from that of the second nanoparticle. In some non-limiting examples, the first nanoparticle may have a shape including at least one vertex, the second nanoparticle may have a shape including at least one edge, and the vertex of the first nanoparticle may be proximate to the edge of the second nanoparticle 120i. In some alternative non-limiting examples, the first nanoparticle may have a shape including at least one vertex, the second nanoparticle may have a shape including at least one vertex, and the vertex of the first nanoparticle may be proximate to the vertex of the second nanoparticle 120j. In embodiments of nanostructures composed of two or more nanoparticles, the nanoparticles may be separated from each other by a distance of about 1 nanometers to about 10 nanometers. Non-limiting examples of such separation distances may include about 1 nanometers, about 2 nanometers, about 3 nanometers, about 4 nanometers, about 5 nanometers, about 6 nanometers, about 7 nanometers, about 8 nanometers, about 9 nanometers, about 10 nanometers, and ranges between any two of these values including endpoints.

As illustrated in FIG. 1, the assay plate 100 may include an arrangement of nanostructures 120a-j on a substrate 110. In some non-limiting embodiments, the nanostructures 120a-j may be arranged on the substrate 110 in an array. Non-limiting examples of such an array may include one or more of a square array, a rectangular array, a linear array, a circular array, and a random array.

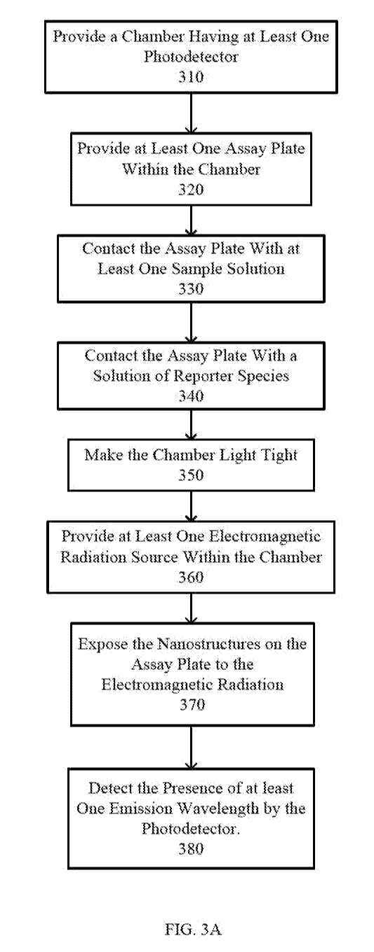

Nanostructures 210 may absorb electromagnetic radiation 220 at distances far greater than the nanoparticle size, as illustrated in FIG. 2A. FIG. 2B illustrates plasmonic field lines 240 generated around a pair of narrowly separated nanoparticles 230. It may be appreciated that the plasmonic field lines 240 may be locally concentrated. This may be observed in the concentrated field 244 located between the tips of the two triangular nanoparticles 230 as well as at the corners 242. It may be appreciated that fluorescent reporter species that bind at these locations may receive a significant amount of plasmonic energy, and consequently emit an intense fluorescence.

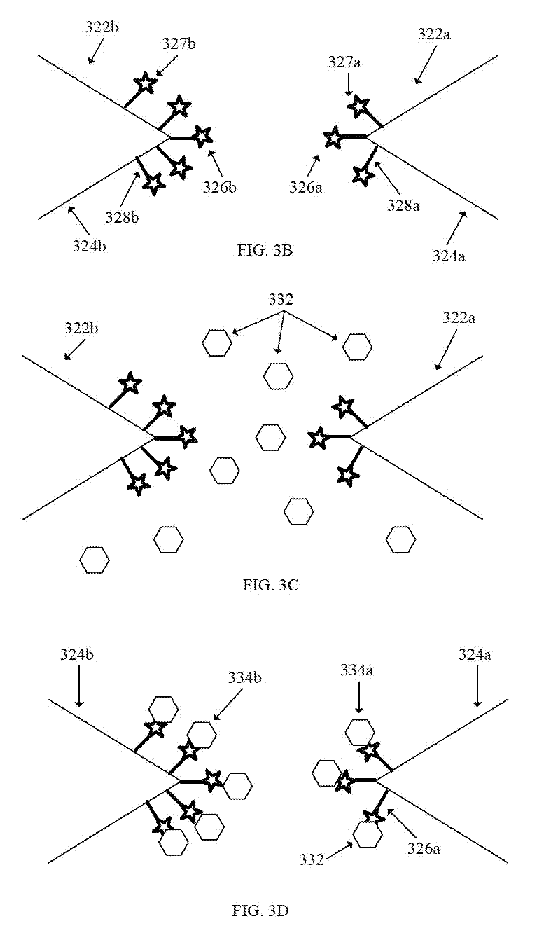

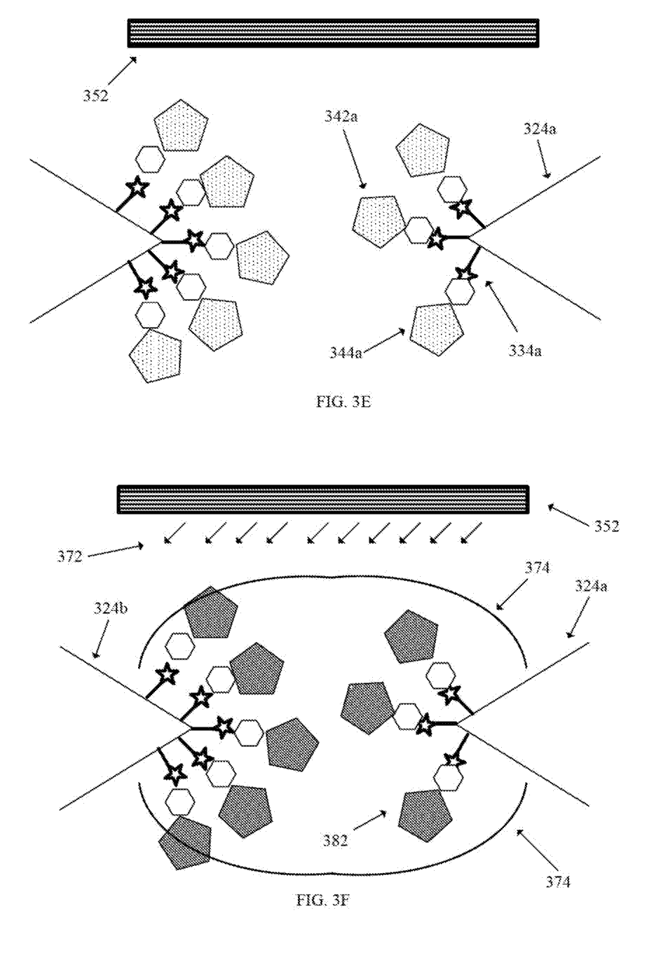

FIG. 3A is a flow chart of an illustrative method of using a PREFI assay to detect one or more biomolecule targets. Since the PREFI assay may include the detection of fluorescent light, an assay chamber that may include at least one photodetector may be provided 310. The chamber may be light tight or at least capable of being placed in a light tight state.

During the assay, at least one assay plate may be provided 320 within the chamber. The chamber may be constructed to allow multiple assay plates to be placed within it, either all at one time, or in a sequential manner The assay plate may be composed of a number of nanostructures in contact with a substrate, as illustrated in FIG. 1. The nanostructures may also incorporate biomolecule capture species. These capture species may be constructed to bind to one type of biomolecule. It may be appreciated that a single assay plate may include a number of nanostructures, all the nanostructures having a single, homogeneous type of capture species designed to bind to a single type of biomolecule. Alternatively, a single assay plate may include one or more groups of nanostructures having one type of capture species designed to bind a first biomolecule, and a second group of nanostructures having a second type of capture species designed to bind a second biomolecule. An assay plate may also incorporate any number of nanostructures, the nanostructures including any number of distinct types of capture species.

The one or more assay plates may be contacted with at least one sample solution 330. In some embodiments of the assay method, contacting the one or more assay plates with at least one sample solution 330 may include providing the at least one sample solution, allowing the one or more assay plates with the sample solution to incubate for a period of time, removing the sample solution, and washing the one or more assay plates with a washing solution, such as aqueous solutions of polysorbate detergents such as TWEEN® (manufactured by Sigma Aldrich Corp., St. Louis, Mo.), thereby removing any unbound material. It may be appreciated that the assay method contemplates that the sample solution may or may not contain the biomolecule to be assayed. It may also be understood that incubating the one or more assay plates in the presence of the sample solution may include, alone or in combination, allowing the one or more assay plates to remain static, manipulating the one or more assay plates to mix the solution over the nanostructures, such as by pumping fluid across the assay surface using microfluidic channels, agitating the assay plates, or subjecting the assay plates to ultrasonic vibrations. Incubating the one or more assay plates may also include maintaining the one or more assay plates at a predetermined temperature, electrochemical potential, or pH.

It may also be understood that additional derivatives, methods, and procedures associated with the range of ELISA-type assaying procedures, alone or in combination, may be included as additional embodiments to those disclose herein. Such additional methods may include, without limitation, sandwich ELISA, double antigen ELISA, and competitive ELISA.

The one or more assay plates may be contacted 340 with one or more solutions containing reporter species. Each type of reporter species may specifically bind to one type of target biomolecule. For simplicity, the combined structure of reporter species bound to a target biomolecule that is further bound to a capture species may together be termed an “assay complex”. Unbound reporter species may be washed away. For a reporter species including a fluorophore, the assay complex may absorb electromagnetic radiation within a band of absorption wavelengths, and may emit electromagnetic radiation at one or more wavelengths within a band of emission wavelengths. It may be appreciated that the absorption band and emission band of the assay complex may be shifted or otherwise differ from the absorption band and the emission band, respectively, of the free fluorophore. In one non-limiting embodiment, contacting the assay plate with the solution of reporter species may further include contacting the one or more assay plates with the reporter species solution, allowing the one or more assay plates with the reporter solution to incubate for a period of time, removing the reporter solution, and washing the one or more assay plates with a washing solution, such as aqueous solutions of polysorbent detergents, thereby removing any unbound material. It may also be understood that incubating the one or more assay plates in the presence of the reporter solution may include, alone or in combination, allowing the one or more assay plates to remain static, manipulating the one or more assay plates to mix the solution over the nanostructures, such as by pumping fluid across the assay surface using microfluidic channels, agitating the assay plates, or subjecting the assay plates to ultrasonic vibrations. Incubating the one or more assay plates may also include maintaining the one or more assay plates at a predetermined temperature, electrochemical potential, or pH.

Once the assay complexes have been formed, the amount of bound target biomolecule may be determined through a fluorescence detection method. For this method, the chamber may be configured 350 into a light tight state so that ambient light may not interfere with the fluorescence detection.

Within the chamber, at least one source of electromagnetic radiation may be provided 360. In some non-limiting embodiments, the electromagnetic radiation may be coherent electromagnetic radiation. In some non-limiting embodiments, the electromagnetic radiation may be polarized electromagnetic radiation. Polarized electromagnetic radiation may be either linearly or circularly polarized. In some non-limiting embodiments, the electromagnetic radiation may be pulsed. The pulses, or short pulse trains, may be in the femtosecond, picosecond, nanosecond, microsecond or millisecond regime. In some non-limiting embodiments, the electromagnetic radiation may be emitting pulses at different wavelengths in synergetic sequences. In some non-limiting embodiments, the pulse sequence may be designed to excite a portion of the assay/assays using a pulse of a certain wavelength, followed by a quenching pulse of another wavelength to enhance some predetermined signal to noise ratio. In some non-limiting embodiments, the electromagnetic radiation may include of a first short high intensity pulse, such as in the femtosecond or picosecond regime, followed by a longer pulse at lower intensity that may also be of a different wavelength.

Typically, for fluorescence processes, a fluorophore may absorb radiation having photons with a wavelength around the fluorophore absorbance peak, thereby placing the fluorophore in an excited state. Intersystem crossing within the fluorophore causes the fluorophore to drop to a lower energy state, and consequently the fluorophore emits light at a longer wavelength than the absorbance wavelength. Because the energy loss through intersystem crossing typically is not great, the fluorophore emission wavelength may not be much greater than the absorption wavelength, and thus difficult to filter. In general, photons having wavelengths greater than the absorbance peak do not have sufficient energy to be absorbed. However, if the flux of the longer-wavelength photons is sufficiently great, for example a flux provided by a plasmonic dipole field, the fluorophore may absorb electromagnetic energy equivalent to a sufficient number of photons effectively simultaneously (a quasi-multi-photon absorption process), thereby allowing the fluorophore to enter its excited state with subsequent emission of fluorescent light. Without being bound by theory, components of the energy flux may flow from the plasmonic field to the fluorescent marker in both the absorption phase as well as the emission phase, so the exact direction of energy flow should not be considered a limiting factor. Further, for the purposes of this disclosure, the entire plasmonic nanoparticle-biomolecule-fluorescent marker species may be considered a single absorption/emission species. For a quasi-multi-photon absorption process, the fluorescence excitation energy (the longer wavelength photons) may be well separated from the emission energy, and thus easier to filter.

In order to take advantage of the greater ease of separating the excitation wavelength from the emission wavelength, associated with quasi-multi-photon absorption processes, the source of electromagnetic radiation may be configured to emit excitation wavelengths greater than the absorption band of the assay complexes. In some embodiments, the emitted excitation wavelengths of the electromagnetic radiation source may be greater than the emission band of the assay complexes. In some non-limiting examples, the electromagnetic radiation may include at least one excitation wavelength that is at least 50% greater than at least one emission wavelength within the emission band of the assay complex. In some non-limiting examples, the electromagnetic radiation may include at least one excitation wavelength that is at least twice as large as at least one emission wavelength within the emission band of the assay complex. In some non-limiting examples, the electromagnetic radiation may include at least one excitation wavelength that is at least three times as large as at least one emission wavelength within the emission band of the assay complex.

At least some portion of the several nanostructures in the one or more assay plates may be exposed 370 to the electromagnetic radiation from the electromagnetic radiation source, thereby inducing the plasmonic dipole fields around (proximal to) the nanostructures. In some non-limiting examples, the electromagnetic radiation may have at least one excitation wavelength about 600 nanometers to about 1550 nanometers. Non-limiting examples of such excitation wavelengths may include about 600 nanometers, about 700 nanometers, about 800 nanometers, about 900 nanometers, about 1000 nanometers, about 1100 nanometers, about 1200 nanometers, about 1300 nanometers, about 1400 nanometers, about 1500 nanometers, about 1550 nanometers, and ranges between any two of these values including endpoints.

It may be appreciated that the nanostructures of the one or more assay plates may be exposed 370 to the electromagnetic radiation according to a number of different methods. For example, all the nanostructures may be exposed 370 to the radiation simultaneously. Alternatively, separate portions of nanostructures (as individual groups or subsets) may be exposed 370 to the radiation sequentially, for example, in accordance with some timed sequence. In yet another alternative procedure, individual nanostructures may be exposed 370 to the radiation sequentially, for example, in accordance with some timed sequence.

Emission radiation, such as a fluorescence signal, from the assay complexes may be detected 380 by the at least one photodetector within the chamber. In one non-limiting example, the emitted radiation may include one or more wavelengths of emitted fluorescent radiation from within the emission band of the assay complexes. In some alternative examples, the emitted radiation may include one or more wavelengths of emitted phosphorescent radiation from within the emission band of the assay complexes.

FIGS. 3B-F present diagrammatically an illustrative method of detecting a target biomolecule according to the flow chart of FIG. 3A. It may be understood that the components, elements, and processes illustrated in FIGS. 3B-F may occur in the chamber previously discussed in reference to FIG. 3A.

FIG. 3B illustrates a pair of nanostructures, 322a,b configured with their respective apices proximal to each other. The nanostructures 322a,b may be composed of nanoparticles 324a,b to which are attached some number of capture species 326a,b. In one non-limiting example, the capture species 326a,b may be composed of one or more biomolecule binders 327a,b in contact with one or more molecular linkers 328a,b. Without being bound by theory, the one or more molecular linkers 328a,b may act to maintain the biomolecule binders 327a,b at a preferred distance from the nanoparticles 324a,b. The biomolecule binders 327a,b may be designed to bind specifically to a type of target biomolecule. The biomolecule binders may be separated on the nanostructure surface by spacer molecules. Without being bound by theory, such spacer molecules may increase the distance between reporter species, thereby reducing quenching effects arising from the close proximity of the chromophores.

FIG. 3C illustrates an example of the nanostructures 322a,b being exposed to a solution including target biomolecules 332. FIG. 3D illustrates an example of target biomolecules 332 binding to the nanostructures 324a,b through the capture species 326a. The combined biomolecule 332 and capture species 326a may be termed a pre-assay complex 334a.

FIG. 3E illustrates an example of pre-assay complexes 334a after exposure to a solution of reporter species 344a. As disclosed above, a specific type of reporter species 344a may be configured to contact a specific type of target biomolecule. FIG. 3E illustrates the nanoparticles 324a contacting the assay complexes 342a composed of pre-assay complexes 334a and the reporter species 344a. It may be noted that a source of electromagnetic illumination 352 is also illustrated in FIG. 3E. For the processes illustrated in FIG. 3E, the source of electromagnetic illumination 352 is off.

FIG. 3F illustrates the result of exposing 370 the nanostructures to the electromagnetic radiation 372 produced by the radiation source 352. As a result of the electromagnetic radiation 372, the nanoparticle 324a,b may produce a plasmonic dipole field 374 that may extend through the gap between the nanoparticles. FIG. 2B illustrates one example of the plasmonic dipole field lines that may be generated by the nanoparticle geometry illustrated in FIG. 3F. Assay complexes 342a within the plasmonic field 374 may experience a high electromagnetic field environment. While the incident electromagnetic radiation 372 may have a wavelength greater than either the absorption band or emission band of the assay complexes, sufficient electromagnetic energy density may be generated at the plasmonic field “hot spot” (see FIG. 2B 244) such that the reporter species located there may be excited and subsequently emit light 382 as a result of quasi-multi-photon absorption processes.

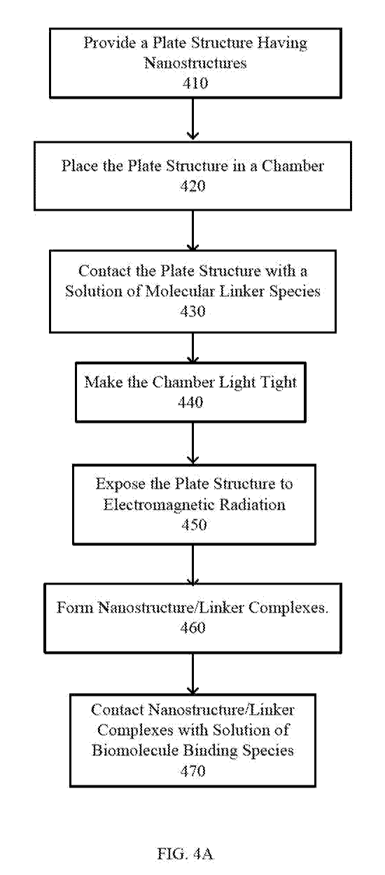

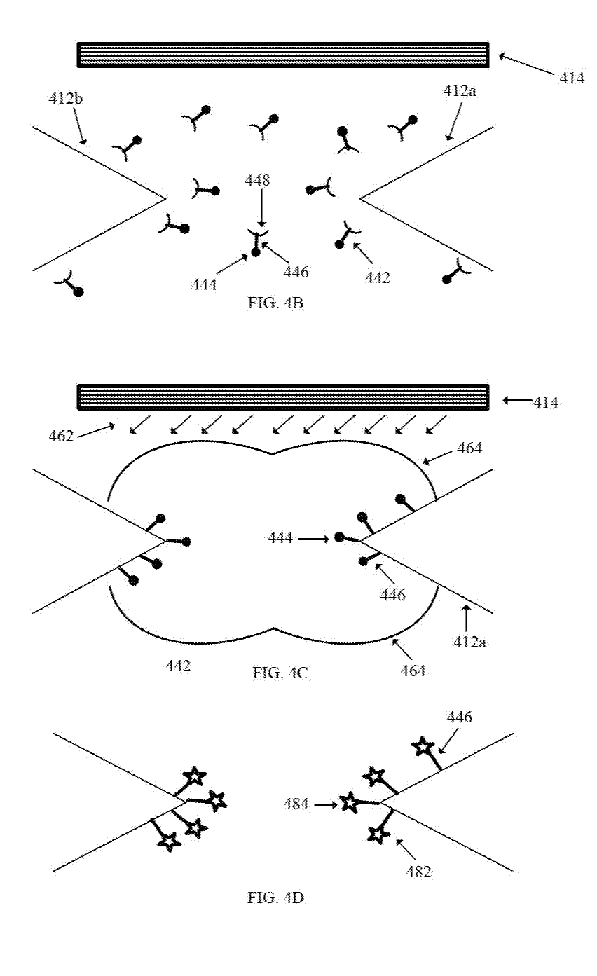

It may be appreciated that proper location of the capture species relative to the nanostructures may be important to assure that the assay complexes receive sufficient field strength from the plasmonic fields. FIG. 4A is a flow chart of one non-limiting example of a method to fabricate an assay plate.

A plate structure having a number of nanoparticles contacting a substrate may initially be provided 410. Descriptions of possible nanoparticle compositions, sizes, and shapes have been disclosed above. Descriptions of possible substrate compositions have also been disclosed above.

In some non-limiting embodiments, the plate structure may be cleaned to remove deposits on the substrate and nanoparticles. Cleaning the plate structure may include one or more of standard RCA wafer cleaning procedures, acid/base etching, piranha solutions (a solution including a combination of sulfuric acid and hydrogen peroxide), rinsing in solvents such as water, alcohols, acetones, detergents or chloroforms. The cleaning may be aided by standard means such as temperature control and sonication. The plate structure may also be coated after cleaning with molecules suitable for subsequent processing. The plate structure, with or without cleaning, may be placed 420 in a chamber that may include at least one illumination source. The chamber may also be configurable to be placed in a light tight state.

Within the chamber, the cleaned plate structure may be contacted 430 with a first solution that may include a molecular linker species. The chamber may be placed in a light tight state 440 to reduce the possibility of extraneous ambient light. The plate structure may be exposed 450 to electromagnetic radiation emitted by the at least one illumination source. The illumination source may generate electromagnetic radiation that may not intrinsically (that is, without the aid of other processes) possess a combination of intensity or wavelength energy capable of enabling significant attachment of the molecular linker species on any assay surface. Without being bound by theory, exposure 450 of the nanoparticles to light of suitable wavelength and intensity may thereby induce at least one plasmon dipole electric field proximal to one or more of the plurality of nanoparticles. Exposing 450 the plate structures to electromagnetic radiation emitted by the at least one illumination source may include exposing the plate structures to electromagnetic radiation, allowing the plate structures to incubate while being illuminated, removing the first solution from contact with the plate structure, and washing the plate structure with a washing solution. It may also be understood that incubating the one or more plate structures in the presence of the first solution may include allowing the one or more plates to remain static, manipulating the one or more plates to mix the solution over the nanoparticles (such as by agitation), or maintaining the one or more assay plates at a predetermined temperature, pH, or electrochemical potential.

In some non-limiting examples, the electromagnetic radiation may be coherent electromagnetic radiation. In some non-limiting examples, the electromagnetic radiation may be polarized electromagnetic radiation. In some non-limiting embodiments, the electromagnetic radiation may be pulsed. The pulses, or short pulse trains, may be in the femtosecond, picosecond, nanosecond, microsecond or millisecond regime. In some non-limiting embodiments, the electromagnetic radiation may be emitting pulses at different wavelengths in synergetic sequences. In some non-limiting embodiments, the pulse sequence may be designed to excite a portion of the structures using a pulse of a certain wavelength, followed by a quenching pulse of another wavelength. In some non-limiting embodiments, the electromagnetic radiation may include a first short high intensity pulse, such as in the femtosecond or picosecond regime, followed by a longer pulse at lower intensity that may also be of a different wavelength. In some non-limiting examples, the electromagnetic radiation may have at least one excitation wavelength about 600 nanometers to about 1550 nanometers. Non-limiting examples of such wavelengths may include about 600 nanometers, about 800 nanometers, about 1300 nanometers, about 1550 nanometers, and ranges between any two of these values including endpoints.

Linker species may contact the nanoparticles at regions of maximal plasmon dipole field values, thereby forming 460 nanoparticle/molecular linker complexes. In some non-limiting embodiments, separate combinations of sources of electromagnetic radiation may operate on individual targets and thereby individually excite (or otherwise transform into a state more suitable for attachment/reaction) one or more of the linker species, reaction catalysts, reagent molecules, surface bound molecules, and plasmonic dipole fields. The linker species may have a terminal group composed of one or more photosensitizers. In some non-limiting examples, the one or more photosensitizers may include a porphyrin dye (including, as an example, tetraphenylporphyrin), a dye, a photo-oxidizable terminal thiol group, a single step photocatalytic species, or a multi-step photocatalytic species. In some non-limiting examples, the method may further include removing the first solution having at least one species of molecular linker from the plate structure and washing the plate structure with a washing solution.

The plate structure, including the nanoparticle/molecular linker complexes may then be contacted 470 with a second solution including at least one species of biomolecule binder. Each type of biomolecule binder may have a specificity for a particular type of target biomolecule. The biomolecule binder may then bind to the linking species, thereby forming a capture species directed to binding to a specific type of target biomolecule. In a non-limiting example, the binding mechanism may be enabled by hexa histidine tagged biomolecules that bind specifically to a linking molecule possessing a Ni-chelate moiety. Assay plates may thus be composed of plate structures having biomolecule capture species associated with the nanoparticles. In some alternative methods, the second solution may be removed from the assay plates, and the assay plates may be washed with a washing solution.

FIGS. 4B-D present diagrammatically one example of fabricating an assay plate according to the flow chart of FIG. 4A. FIGS. 4E-F present diagrammatically a second example of fabricating an assay plate. It may be understood that the components, elements, and processes illustrated in FIGS. 4B-F may occur in the chamber previously discussed in reference to FIG. 4A.

FIG. 4B illustrates a pair of nanoparticles 412a,b configured with their respective apices proximal to each other. The nanoparticles 412a,b may be associated with a substrate (not shown) that together may form a plate structure. The plate structure may be placed in a chamber also including an illumination source 414. In FIG. 4B, the illumination source is off and does not provide any electromagnetic radiation. The chamber may also be configured to be in a light tight state. The nanoparticles 412a,b may be contacted with a solution including at least one species of molecular linker 442. The molecular linker 442 may be composed of a head component 444, a spacer component 446, and a tail component 448.

FIG. 4C illustrates the effect of irradiating the nanoparticles, such as nanoparticle 412a, with electromagnetic radiation 462 provided by illumination source 414. The nanoparticles may transfer energy from the plasmonic dipole fields 464 as a result of the electromagnetic illumination. Under the influence of the plasmonic dipole field 464, the molecular linkers 442 may associate with the nanoparticles, such as 412a, in the region in which the plasmonic dipole field 464 is strong. Without being bound by theory, under the influence of a strong plasmonic dipole field 464, the tail component 448 of the molecular linkers 442 may react with the surface of the nanoparticles 412a in such a manner as to permit the spacer component 446 to associate with or bind to the nanoparticle surface. Examples of photochemical reactions that may be enhanced under the influence of photonic dielectric fields may include one or more of Norrish reactions, the use of photosensitizers such as a porphyrin (an example including tetraphenylporphyrin) or dyes such as methylene blue, photo-oxidation of terminal thiols, and other single step or multi-step photocatalytic reactions. The association process, in one non-limiting process, may be aided by the establishment of an electrochemical potential between the tips. In some embodiments, the tail components 448 of the molecular linker may be lost as part of the association reaction. However, the molecular linker 442 may not react with the surface of the nanoparticle 412a in areas not under the influence of a strong plasmonic field 464. Thus, the photoactivated end of a molecular linker 442 may become inert if it diffuses outside the high dipole field before it associated itself to the surface. In this manner, the molecular linkers 442 may be localized only on the surface of nanoparticles 412a that may experience strong plasmonic fields 464. As illustrated in FIG. 2B 244, such a strong field may be generated in a region between the apices of two closely spaced nanoparticles. As a result, a nanoparticle/molecular linker complex may be composed of a portion of the nanoparticle 412a surface, the spacer components 446, and the head components 444.

The head component 444 may be selected to specifically associate with a biomolecule binder 484. The plate structure may be exposed to a second solution including the biomolecule binder 484. An example of the result of the association of the biomolecule binder 484 with the head component 444 is illustrated in FIG. 4D. It may be appreciated that the combination of biomolecule binder 484 with the head component 444 and the spacer component 446 together form the biomolecule capture species 482 (see also 326a,b in FIG. 3B). In turn, the biomolecule capture species 482 together with the nanoparticles 412a may form the nanostructures (see 322a,b in FIG. 3B). An assay plate may thus be composed of the nanostructures (from the nanoparticles 412a and their associated capture species 482) together with the substrate on which the nanoparticles reside.

FIGS. 4E and 4F illustrate an alternative example of a method for fabricating an assay plate. FIG. 4E appears similar to FIG. 4B except the molecular linkers 442 in FIG. 4B are replaced with a pre-molecular linker 492. The pre-molecular linker 492 may be composed of a tail component 448, a spacer component 446, and a biomolecule binder 484. The biomolecule binder 484 may be directly associated with the spacer component 446 or it may form an association with a head component (not shown) initially coupled to the spacer component 446.

FIG. 4F illustrates an example of the effect of illuminating the nanoparticles 412a,b in the presence of the pre-molecular linkers 492 with electromagnetic radiation 462 provided by an illumination source 414. The nanoparticles 412a,b under illumination 462 may generate a plasmonic dipole field 464. The pre-molecular linkers 492 may associate with surface of the nanoparticles 412a,b under the influence of the plasmonic dipole field 464 in a manner analogous to the molecular linkers 442. The resulting assay plate may be composed of the substrate and associated nanoparticles 412a,b contacting the capture species 482.

While a single sequence of steps have been disclosed above with respect to fabricating an assay plate having specificity to a single biomolecule, it may be appreciated that a single plate with specificities to multiple biomolecules may be formed. As a non-limiting example, a first subset of the nanoparticles may be illuminated and exposed to a first molecular linker specific to a first biomolecule binder, thereby forming a first capture species. A second subset of the nanoparticles may be illuminated and exposed to a second molecular linker specific to a second biomolecule binder, thereby forming a second capture species. In this manner, an assay plate may be fabricated to detect more than one biomolecule.

In another embodiment, the substrate of the assay plate may be pre- or post-coated with one or more agents to prevent non-specific binding of samples. Such agents may include albumin or other agents to prevent non-specific sample binding.

In another non-limiting embodiment, the molecular linkers or pre-molecular linkers may be associated with the nanostructures solely through electrochemical reactions as opposed to through the use of the plasmonic dipole fields. Electrochemical reactions may be induced through the application of an electric field to the nanostructures. Such an electric field by be applied through a power source connected directly to the nanostructures or through integrated circuitry associated with the nanostructures.

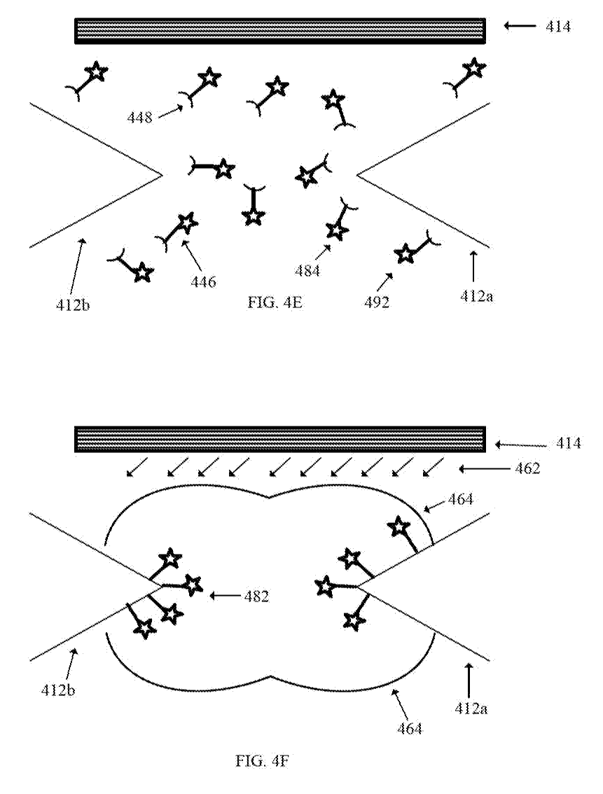

FIG. 5 illustrates a non-limiting example of a system 500 that may be used to detect a biomolecule. The system may include at least one assay plate 510, a chamber 520 in which the at least one assay plate may be analyzed, at least one illumination source 530, and at least one photodetector 540. The assay plate 510 may be composed of a substrate 514 on which are disposed a number of nanostructures 512. The nanostructures 512 may further include nanoparticles in contact with one or more biomolecule capture species that may be configured to bind the biomolecule of interest. The chamber 520 may be configurable to be placed in a light tight state. The illumination source 530 may further be configured to emit electromagnetic radiation 532 having at least one excitation wavelength able to induce a plasmonic dipole electric field proximal to one or more of the plurality of nanostructures 512.

The nanostructures 512 may have an average length of about 30 nanometers to about 1500 nanometers. Non-limiting examples of the average length of a nanostructure may include about 30 nanometers, about 50 nanometers, about 100 nanometers, about 200 nanometers, about 300 nanometers, about 400 nanometers, about 500 nanometers, about 600 nanometers, about 700 nanometers, about 800 nanometers, about 900 nanometers, about 1000 nanometers, about 1250 nanometers, about 1500 nanometers, and ranges between any two of these values including endpoints. In some non-limiting examples, the nanostructures 512 may have an average length less than or equal to about one half of at least one excitation wavelength. In some non-limiting examples, the nanostructures 512 may have an average length less than or equal to about one third of at least one excitation wavelength. In some non-limiting examples, the nanostructures 512 may have an average length less than or equal to about one fifth of at least one excitation wavelength. In some non-limiting examples, the nanostructures 512 may have an average length less than or equal to about one tenth of at least one excitation wavelength. In some non-limiting examples, the nanostructures 512 may have an average length less than or equal to about one hundredth of at least one excitation wavelength.

The nanostructures 512 may include one or more types of biomolecule capture species, at least some group of which may be specific to binding a specific type of biomolecule. Non-limiting examples of the biomolecule capture species may include one or more of a polyvalent species, a chelating species, a biomolecule antibody, a nucleic acid, and a protein binding species.

One or more photodetectors 540 may be included within the chamber 520. The at least one photodetector 540 may include one or more of a photoconductor, a photoresistor, a photodiode, a phototransistor, a quantum dot photoconductor, a photodiode array, an avalanche photodiode, and a CCD array. It may be understood that multiple photodetectors 520 may be included, and that the multiple photodetectors may be of the same type or different types. In one example, different photodetectors 520 may be used, each having a sensitivity to a different wavelength. The photodetectors 520 may also include additional optical components including optical filters, polarizers, and lenses to improve sensitivity to radiation emitted by reporter species bound to captive biomolecules.

The one or more illumination sources 530 may include, without limitation, one or more of a broad-band light source, a narrow-band light source, a coherent light source, a polarized light source, a tunable light source, a laser, a tunable laser, a pulsed laser, a laser diode, an incandescent light, a fluorescent light, and a high pressure gas light source. The at least one illumination source 530 may be configured to source electromagnetic radiation 532 having at least one wavelength in one or more of the near IR spectrum, the far IR spectrum, the visible spectrum, and the near UV spectrum.

In some non-limiting embodiments, the at least one illumination source 530 may be configured to illuminate substantially all the nanostructures 512 in the array of nanostructures simultaneously. Alternatively, the at least one illumination source 530 may be configured to illuminate a number of individual portions of the array of nanostructures 512 sequentially. As a non-limiting example, the illumination source 530 may include a laser that can sequentially illuminate small blocks of, or even individual, nanostructures 512 in a sequential manner In such an example, the laser illumination 532 may be directed onto the assay plate 510 by controllable optical components, such as by reciprocating mirrors or rotating prisms. Alternatively, the laser itself may be moved by means of a controllable actuator (not shown).

In some non-limiting examples, the at least one illumination source 530 may be configured to direct illumination orthogonally to a plane comprising the array of nanostructures 512. Such a plane may be defined by the assay plate 510 or substrate 514. In still another non-limiting example, the at least one illumination source 530 may be configured to direct illumination at an angle that is not orthogonal to a plane comprising the array of nanostructures 512. Such a configuration may be useful to minimize the amount of scattered illumination 532 from the assay plate 510.

Although not shown in FIG. 5, it may be appreciated that the detection system 500 may further include one or more electronic components to form a control system to control the operation of the system and provide results of analyzing the one or more assay plates. Although specific components of such a control system may be disclosed below, such components should neither be considered as limiting nor required for any such detection system 500.

A system bus may serve as the main information highway interconnecting the other controller hardware components. At least one CPU, the central processing unit of the system, may perform calculations and logic operations required to execute a program to control the system 500. Read only memory (ROM) may be one example of a static or non-transitory memory device, and random access memory (RAM) may be one example of a transitory or dynamic memory device. A disc controller may interface the system bus with one or more optional disk drives. These disk drives may include, for example, external or internal DVD drives, CD ROM drives, or hard drives.

Program instructions, to control the one or more illumination sources 530 and one or more photodetectors 540 may be stored in the ROM and/or the RAM. Such instructions may include control of laser pulse operations for pulsed lasers, laser output wavelengths for tunable lasers, positioning of movable optical elements for either the one or more illumination sources 530 and/or photodetectors 540. Additional program instructions related to analyzing the output of the one or more photodetectors 540 may be stored as well. Optionally, program instructions may be stored on a computer readable medium such as a compact disk or a digital disk or other non-transitory recording medium.

A display interface may permit information from the bus to be provided to a user in audio, graphic or alphanumeric format. Some non-limiting examples of some output or display devices may include a printer, an LCD panel device, a touch screen device, an audio device, an LED panel, and an OLED panel device. Communication between the system controller and external devices may also occur over various communication ports.

The controller system may also include input devices such as a keyboard. Alternative input devices may include one or more of a touch screen, a mouse, and/or a joystick. Input devices may permit a user to provide specific control instructions to the controller. Such control instruction may include control of the one or more sources of illumination 530, control of the one or more photodetectors 540, control of additional optical elements (filters, lenses), motion control of the one or more assay plates 510 within the chamber 520, control of the placement of the one or more assay plates 510 within the chamber 520, or similar controls. Input devices may also permit a user to control the manner in which photodetector 540 data are analyzed and presented to the user.

A system such as that disclosed above may also be used with one or more kits for detecting one or more biomolecules. It is contemplated that a user may purchase a detecting system that can readily be adapted to detect any of a number of different biomolecules depending on the kit used with the detecting system.

In some non-limiting embodiments, a kit may include at least one assay plate, a source of a first solution including at least one binder species configured to bind to the biomolecule, a source of a second solution comprising at least one molecular linker species, and at least one source of a third solution including a reporter species configured to bind to the biomolecule. The assay plate may have a number of nanostructures disposed on a substrate. The nanostructures may be composed of a number of pairs of nanoparticles, each nanoparticle having at least one vertex. In a non-limiting example, the vertex of the first nanoparticle of the pairs of nanoparticles may be proximate to the vertex of the second nanoparticle of the pairs of nanoparticles. Both the binder species and the reporter species may have at least some specificity for binding to the same biomolecule. The linker species may have a first end configured to contact at least one of the several nanoparticles and a second end configured to contact at least one binder species. In addition, the first end of the linker species may be configured to contact a surface of one of the nanoparticles when the nanoparticle is illuminated by electromagnetic radiation having at least one excitation wavelength that is greater than an average length of the nanoparticles.

It may be understood that in addition to the solutions disclosed above, additional sources of solutions may be included in the kit, for example sources for one or more washing solutions.

The first solution, including the binder species, may be an aqueous solution, or a buffered aqueous solution. Depending on the nature of the binder species, other solvents may be used. The binder species themselves may include one or more of a polypeptide, a protein, an enzyme, an antigen, an antibody to the biomolecule, a polynucleotide, at least a portion of an RNA molecule, and at least a portion of a DNA molecule.

The second solution, including the linker species, may be an aqueous solution, or a buffered aqueous solution. Depending on the nature of the linker species, other solvents may be used. The linker species may include one or more chromophores. Non-limiting examples of such chromophores may include conjugated pi-bond systems, fluorophores, and molecules configured to participate in Forster and Dexter energy transfer events. Without being bound by theory, such chromophores may permit a wavefunction overlap between the plasmonic field and the fluorescent reporter species. The distal end of the linker species may include one or more of a polyvalent species, a chelating species, a biomolecule antibody, a nucleic acid, and a protein binding species.

The third solution, including the reporter species, may be one of an aqueous solution and a buffered aqueous solution. Depending on the nature of the reporter species, other solvents may be used. In some non-limiting examples, the reporter species may include one or more of a polypeptide, a protein, an enzyme, an antibody to the biomolecule, a polynucleotide, a quantum dot, at least a portion of an RNA molecule, and at least a portion of a DNA molecule. In some non-limiting embodiments, the reporter species may include one or more fluorescent proteins. In some non-limiting embodiments, the reporter species may include one or more quantum dots. In some non-limiting embodiments, the reporter species may include a fluorescent moiety. Non-limiting examples of such fluorescent moiety may include one or more of a xanthene, a cyanine, a naphthalene, a coumarin, an oxadiazole, a pyrene, an oxazine, an acridine, an arylmethine, and a tetrapyrrole.

The use of the disclosed PREFI assay in a home environment may result in both the system and kit being as robust as possible. A system to be used with a kit may be substantially similar to the one disclosed above, including as well receptacles for vials containing the various solutions. Pumps or syringe activated devices may be used to withdraw the various solutions, and ports may be available to plug into the assay plate to transport the various solutions and samples.

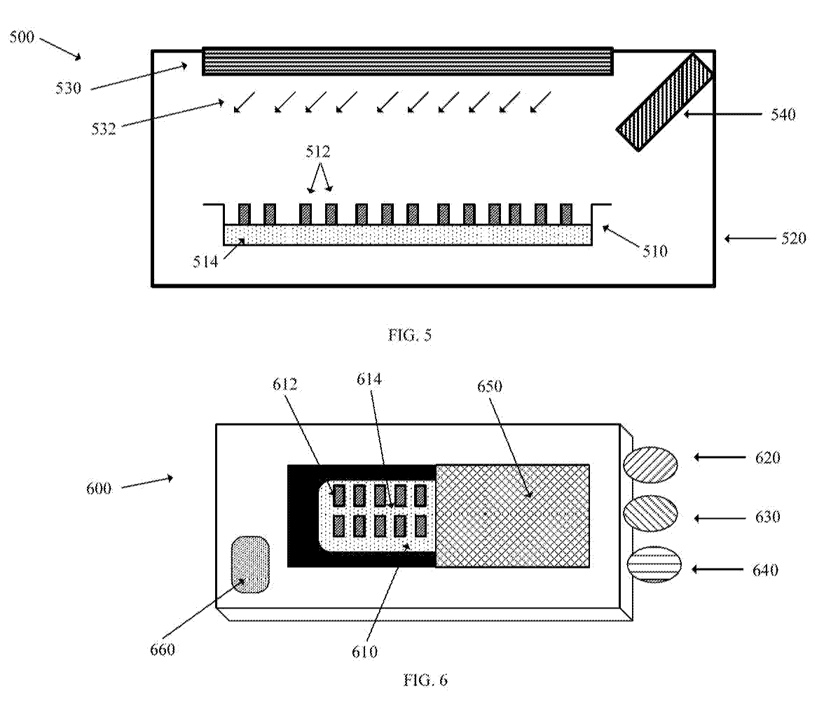

FIG. 6 illustrates an example of a plate enclosure 600 that may be used to insert an assay plate into such a system. The plate enclosure 600 may include at least one assay plate 610 composed of the nanostructures 612 disposed on a substrate 614. The plate enclosure 600 may include one or more assay fluid access ports for the solutions. In one non-limiting example, a common assay fluid access port 620 may permit introduction of any of the first, second, and third solutions into the plate enclosure 600. Alternatively, each of the solutions may be introduced into the plate enclosure 600 via its own separate assay fluid access port. One or more sample access ports 630 may also be provided to allow separate introduction of the sample into the plate enclosure 600. One or more separate wash solution access ports 640 may be dedicated to introducing washing solutions. The solutions within the plate enclosure 600 may be withdrawn through a common withdrawal port, separate withdrawal ports (for possible re-use of the solutions), or through the port used to introduce the solutions. For ease of use, the ports 620, 630, and 640, may be configured to “snap-in” to mating connectors within the system chamber when the plate enclosure 600 is inserted into the system. Such mating connectors may include press-fit or Luer-type connectors, as two non-limiting examples.

One or more microfluid channels (not shown) may be in fluid communication with the one or more assay fluid access ports 620 on one end, and the assay plate 610 on another. In one non-limiting example, such a microfluid channel may have multiple channel entries, each entry in fluid communication with a separate port, including the one or more assay fluid access ports 620 and the one or more sample access ports 630. It is reasonable to have such microfluid channels configured to be in fluid communication with the one or more washing solution access ports 640 as well. Such microfluid channels may be useful to reduce the amount of solution needed for fabricating the assay plate or conducting the assay.

The plate enclosure 600 may also be equipped with a light-tight retractable cover 650. The light tight retractable cover 650 may be manipulated by the system to permit exposure of the assay plate 610 to illumination during illuminations processes, while maintaining the assay plate in darkness when it is necessary.

An identification mark 660 may also be included with the assay plate enclosure. Such an identification mark 660 may be useful for quality control purposes and may include information such as part number, serial number, lot number, manufacturing date, and other identifiers. In some non-limiting examples, the identification mark 660 may be composed of an electromagnetic radiation detectable mark. In some non-limiting examples, the identification mark 660 may be composed of one or more of a bar-code, a QR code, and an RFID chip. It may be contemplated that an RFID chip may be both readable and writable, and that results or other data obtained by the PREFI assay system may be transferred to the RFID chip as a record of the results obtained from that assay plate.

### EXAMPLES

### Example 1

### A Method for Fabricating an Assay Plate

An assay plate includes a number of dual-triangular nanoparticles. The dual-triangular nanoparticles may be configured in a vertex-to-vertex geometry. The nanoparticles are made from single gold crystals and disposed on a substrate. The nanoparticles are coated with a molecule suitable for further processing. The nanoparticles are exposed to a solution of molecular linker species and illuminated by electromagnetic radiation resulting in linker species being bound to the nanoparticles primarily at regions of maximal plasmonic electric field power. The solution is removed and a solution of a binder species contacts the nanostructures. The binder species bind to the molecular linker species. One example of a molecular linker includes a tail group composed of a light sensitive thiol group and a head end containing a Ni chelate. The light sensitive thiol group acts as a leaving group to allow the remainder of the linker species to bind the linker to the surface of the nanoparticle during illumination. A synthetic antibody to the target biomolecule, containing a hexa-histidine group, binds to the linker through ionic coupling of the hexa-histidine group to the Ni chelate group.

### Example 2

### A Method for Detecting a Biomolecule

An assay plate, as disclosed in Example 1, is exposed to a serum sample in a biomolecule detection system that includes a pumping system. The pumping system pumps the serum sample through microchannels from a sample reservoir to the assay plate. Residual serum is recycled by feeding micro channels back into the sample reservoir. The sample can have reporter species pre-bound to any biomolecules in the sample. The plate is illuminated by light having electromagnetic radiation of about 1550 nanometers. Under the illumination, the reporter species fluoresces. The fluorescence from the plate is measured by a detector. For 2D sensor arrays, the entire plate is illuminated; for single point arrays, the illumination is provided by a highly collimated light source, such as a laser that may be scanned over the surface of the assay plate.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated in this disclosure, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used in this disclosure is for the purpose of describing particular embodiments only, and is not intended to be limiting.

With respect to the use of substantially any plural and/or singular terms in this disclosure, those having skill in the art can translate from the plural to the singular and/or from the singular to the plural as is appropriate to the context and/or application. The various singular/plural permutations may be expressly set forth in this disclosure for sake of clarity.

It will be understood by those within the art that, in general, terms used in this disclosure, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at” least one and one or more to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases one or more or “at” least one and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed in this disclosure also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed in this disclosure can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

From the foregoing, it will be appreciated that various embodiments of the present disclosure have been described for purposes of illustration, and that various modifications may be made without departing from the scope and spirit of the present disclosure. Accordingly, the various embodiments disclosed are not intended to be limiting, with the true scope and spirit being indicated by the following claims.

1. A method for detecting one or more biomolecules, the method comprising:
providing a chamber comprising at least one photodetector;
providing, within the chamber, at least one assay plate having a plurality of nanostructures comprising a plurality of biomolecule capture species;
contacting the assay plate in the chamber with at least one sample solution suspected of containing the one or more biomolecules;
contacting the assay plate in the chamber with an assay solution comprising a plurality of biomolecule reporter species, wherein the plurality of biomolecule reporter species, when bound to the one or more biomolecules associated with the plurality of biomolecule capture species, is configured to emit at least one emission wavelength of radiation;
configuring the chamber into a light-tight state;
providing, within the chamber, at least one source of electromagnetic radiation configured to produce electromagnetic radiation having at least one excitation wavelength greater than the at least one emission wavelength;
exposing at least a portion of the nanostructures on the assay plate to the electromagnetic radiation, thereby inducing at least one plasmon dipole electric field proximal to the exposed nanostructures; and
detecting, by the at least one photodetector, the presence of the at least one emission wavelength.

2. The method of claim 1, wherein the plurality of biomolecule capture species comprises at least one type of capture species configured to bind to at least one type of biomolecules.

3. The method of claim 1, wherein the plurality of biomolecule reporter species comprises at least one type of reporter species configured to bind to at least one type of biomolecules.

4.-5. (canceled)

6. The method of claim 1, wherein the electromagnetic radiation is selected from the group consisting of coherent and polarized electromagnetic radiation.

7. (canceled)

8. The method of claim 1, wherein exposing at least a portion of the nanostructures on the assay plate to the electromagnetic radiation comprises exposing to electromagnetic radiation comprising at least one excitation wavelength that is at least 50% greater than the at least one emission wavelength.

9.-10. (canceled)

11. The method of claim 1, wherein exposing at least a portion of the nanostructures on the assay plate to the electromagnetic radiation comprises exposing to electromagnetic radiation comprising at least one excitation wavelength selected from the group consisting of about 600 nanometers, about 800 nanometers, about 1,300 nanometers and about 1,550 nanometers.

12.-15. (canceled)

16. The method of claim 1, wherein exposing at least a portion of the nanostructures on the assay plate to the electromagnetic radiation comprises exposing a first portion of the plurality of nanostructures to the electromagnetic radiation and exposing a second portion of the plurality of nanostructures to the electromagnetic radiation.

17. The method of claim 1, wherein detecting the presence of the at least one emission wavelength comprises detecting the presence of a fluorescence signal.

18.-27. (canceled)

28. A system for detecting one or more biomolecules, the system comprising:
at least one assay plate comprising a plurality of nanostructures contacting a substrate, wherein at least a portion of the plurality of nanostructures comprises a biomolecule capture species configured to bind to the biomolecule; and
a chamber comprising at least one illumination source and at least one photodetector, wherein the chamber is configurable to be placed in a light-tight state, and the at least one illumination source is configured to emit electromagnetic radiation having at least one excitation wavelength configured to induce a plasmon dipole electric field proximal to one or more of the plurality of nanostructures.

29. The system of claim 28, wherein the plurality of nanostructures comprises one or more of: a metal, a metal alloy, a doped semiconductor, an undoped semiconductor, and a carbon nanostructure.

32.-32. (canceled)

33. The system of claim 28, wherein the plurality of nanostructures comprises graphene.

34. (canceled)

35. The system of claim 28, wherein the substrate comprises a dielectric material.

36. (canceled)

37. The system of claim 28, wherein the biomolecule capture species comprises one or more of a polyvalent species, a chelating species, a biomolecule antibody, a nucleic acid, and a protein binding species.

38. The system of claim 28, wherein the at least one illumination source comprises one or more of a broad-band light source, a narrow-band light source, a coherent light source, a polarized light source, a tunable light source, a laser, a tunable laser, a pulsed laser, a laser diode, an incandescent light, a fluorescent light, and a high pressure gas light source.

39. The system of claim 28, wherein the at least one illumination source is configured to source illumination having at least one wavelength in one or more of the near IR spectrum, the far IR spectrum, the visible spectrum, and the near UV spectrum.

40. The system of claim 28, wherein the at least one photodetector comprises one or more of a photoconductor, a photoresistor, a photodiode, a phototransistor, a quantum dot photoconductor, a photodiode array, an avalanche photodiode, and a CCD array.

41. The system of claim 28, wherein the plurality of nanostructures is selected from the group consisting of a plurality of single nanoparticles and a plurality of pairs of nanoparticles.

42.-49. (canceled)

50. The system of claim 28, wherein the plurality of nanostructures are arranged as an array on the substrate.

51. (canceled)

52. The system of claim 50, wherein the at least one illumination source is configured to illuminate all the nanostructures in the array of nanostructures simultaneously.

53. The system of claim 50, wherein the at least one illumination source is configured to illuminate a plurality of portions of the array of nanostructures sequentially.

54. The system of claim 50, wherein the at least one illumination source is configured to direct illumination orthogonally to a plane comprising the array of nanostructures.

55. (canceled)

56. The system of claim 28, wherein the at least one illumination source comprises a pulsed illumination source.

57. (canceled)

58. The system of claim 28, wherein the at least one illumination source comprises a plurality of illumination sources, wherein each of the plurality of illumination sources emits illumination at an independent illumination wavelength.

59. (canceled)

60. The system of claim 56, wherein the pulsed illumination source is configured to emit at least one sequence of excitatory pulses at a first wavelength and at least one sequence of quenching pulses at a second wavelength.

61. The system of claim 56, wherein the pulsed illumination source is configured to emit a first illumination pulse having a first intensity and a first pulse width and a successive second illumination pulse having a second intensity and a second pulse width.

62. The system of claim 61, wherein the first pulse width is less than or equal to the second pulse width, and the first intensity is greater than or equal to the second intensity.

63. The system of claim 61, wherein the first pulse width is greater than or equal to the second pulse width, and the first intensity is less than or equal to the second intensity.

64.-83. (canceled)
